(19) United States
(12) Patent Application Publication
(10) Pub. No.: US 2003/0114853 A1
(43) Pub. Date: Jun. 19, 2003

(54) Polyaxial cross connector

(76) Inventors: Ian Burgess, Barrington, RI (US); J. Riley Hawkins, Cumberland, RI (US); Christopher Ramsay, New Bedford, MA (US); Frank Bono, Rehoboth, MA (US); Conor McCrea, Brookline, MA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/262,502

(22) Filed: Oct. 1, 2002

Related U.S. Application Data
(60) Provisional application No. 60/328,748, filed on Oct. 12, 2001.

(51) Int. Cl. A61B 17/70

(52) U.S. Cl. 606/61

(57) ABSTRACT

A cross connector for connecting two longitudinal members, such as rods, in spinal surgery has a connectors for connecting to the longitudinal members and a ball joint therebetween to allow polyaxial rotation of the connectors.

This application claims priority from U.S. patent application Ser. No. 60/328,748 filed Oct. 12, 2001.

### FIELD OF THE INVENTION

This invention relates generally to spinal instrumentation and more particularly to an apparatus and method for making connections between two spinal rods.

### BACKGROUND

Typical spinal surgery employs screws anchored into adjoining vertebrae and longitudinal members therebetween to thus stabilize a position of the vertebrae with respect to each other. The longitudinal members may comprise plates or rods. Typically two such longitudinal members are employed, one on either side of the vertebrae. Stability is further enhanced through application of one or more transverse cross connectors connecting the two longitudinal members. A typical example is shown in U.S. Pat. No. 5,522,816 to DiNello et al., incorporated herein by reference.

### SUMMARY OF INVENTION

A cross connector for linking longitudinal members engaged to a spine comprises a first connector for attaching to a first one of the longitudinal members, a second connector for attaching to a second one of the longitudinal members and a linkage between the first and second connectors. At least one polyaxial joint is located between the first and second connectors.

Either one or both of the first and second connectors can comprise a polyaxial joint. Preferably, the first and second connectors comprise a first clamping member and a second clamping member, and a fastener adapted to hold the first clamping member and second clamping member tightly together whereby to grasp one of the longitudinal members. In one aspect of the invention, at least a portion of the polyaxial joint is disposed between the first clamping member and the second clamping member whereby when the fastener holds the first clamping member and second clamping member tightly together, that portion is squeezed to prevent rotation of the polyaxial joint. Preferably, the first clamping member has a first surface adapted to engage one of the longitudinal members and the second clamping member has a second surface adapted to engage one of the longitudinal members with the fastener located between the portion of the polyaxial joint and the first and second surfaces.

In one aspect of the invention, the polyaxial joint comprises a convex surface portion on the linkage and a bearing surface on one of the first and second connectors. The convex portion is preferably spherical.

Preferably, the linkage is curved whereby to arch over a patient's spine. It can comprise a first transverse member and a second transverse member connected in sliding relationship to each other whereby to alter a length of the linkage. Splines and grooves are preferably provided on the mating surfaces thereof to allow sliding and which fit together in an interference fit when compressed together whereby to prevent sliding of the first and second transverse members with respect to each other.

In one aspect of the invention, the polyaxial joint comprises a curved surface connected to either the first connector or second connector and a mating surface thereto connected to the other of the first connector or second connector. The curved surface and mating surface are adapted for movement in three degrees of freedom over one another.

Preferably, the curved surface comprises a convex surface and the mating surface is a complementary concave surface. to fix the convex surface to the concave surface a first threaded aperture penetrates the convex surface, a second aperture is proved through a portion of the cross connector bearing the concave surface and a threaded connector passes through the second aperture and threads into the first aperture. Preferably, the second aperture is wider than the threaded connector.

In one aspect of the invention, a camming member traps one of the longitudinal members into one of the connectors.

### BRIEF DESCRIPTION OF THE DRAWINGS

FIG. 1 is a perspective view of a first embodiment of a cross connector according to the present invention;

FIG. 1A is a sectional view taken along lines 1A-1A of FIG. 1;

FIG. 2 is a top plan view of the cross connector of FIG. 1;

FIG. 3 is a sectional view taken along lines 3-3 of FIG. 2;

FIG. 4 is a top plan view of a second embodiment of a cross connector according to the present invention;

FIG. 5 is a section view taken along lines 5-5 of FIG. 4;

FIG. 6 is a perspective view of a third embodiment of a cross connector according to the present invention;

FIG. 7 is a front elevation view of a fourth embodiment of a cross connector according to the present invention;

FIG. 8 is a front elevation view of a fifth embodiment of a cross connector according to the present invention;

FIG. 9 is a front elevation view of a sixth embodiment of a cross connector according to the present invention;

FIG. 10 is a perspective view of a sixth embodiment of a cross connector according to the present invention;

FIG. 11 is a top plan view of the cross connector of FIG. 10;

FIG. 12 is a sectional view taken along lines 1313 of FIG. 11;

FIG. 13 is a perspective view of a seventh embodiment of a cross connector according to the present invention;

FIG. 14 is an underside view of an alternative locking mechanism for a cross connector according to the present invention; and

FIG. 15 is a sectional view taken along lines 15-15 of FIG. 14.

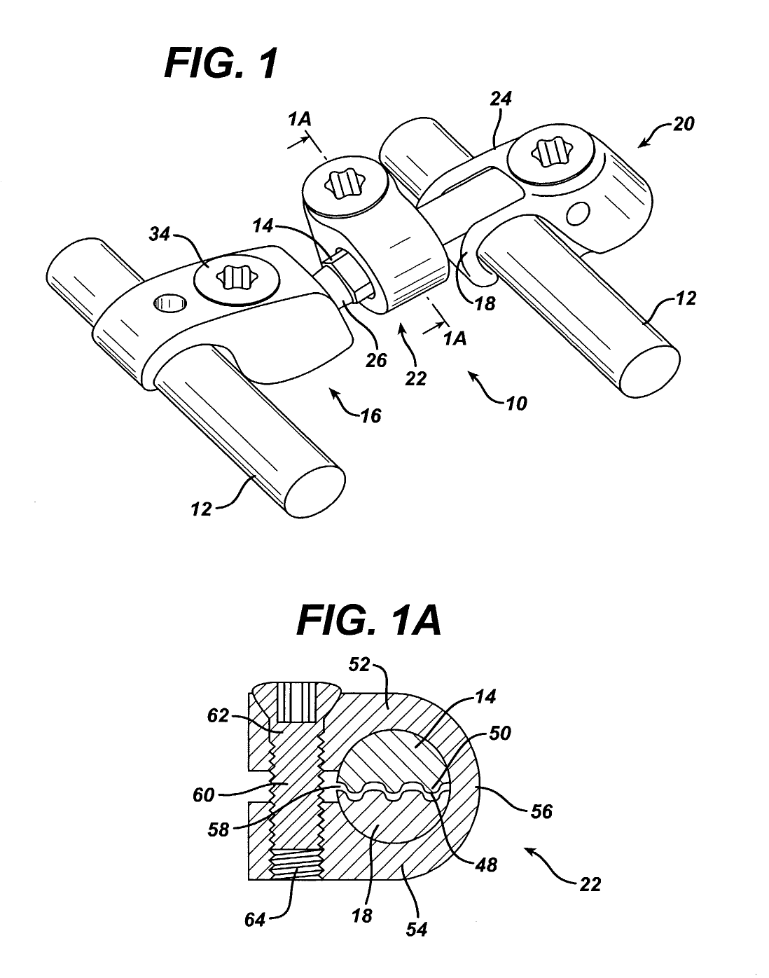

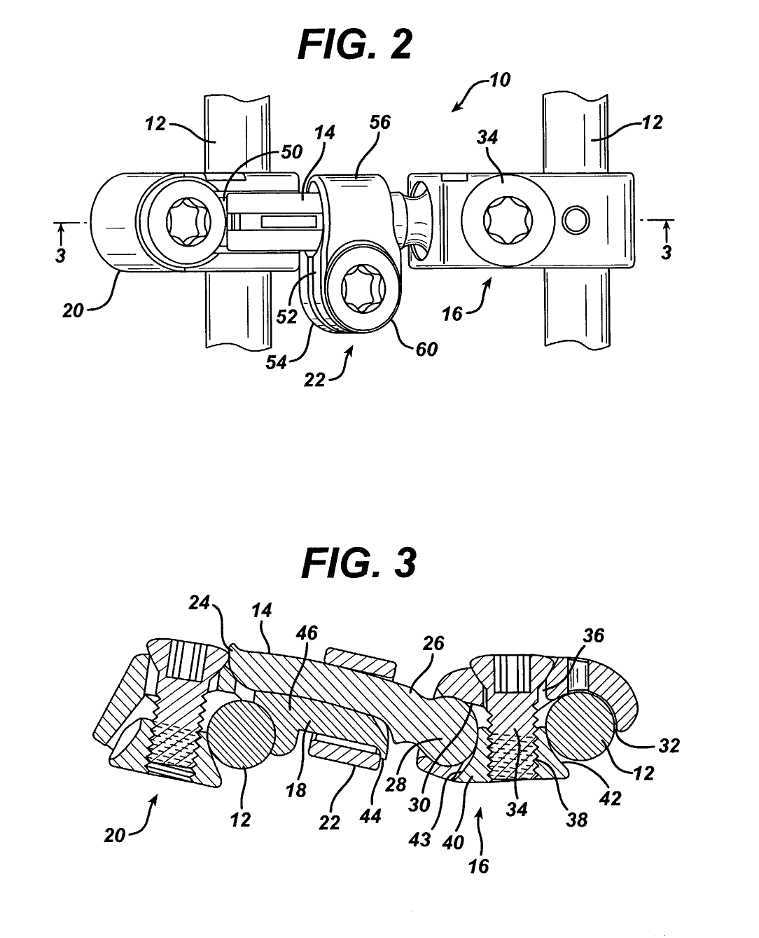

### DETAILED DESCRIPTION

1 to three illustrate a first embodiment of a cross connector 10 according to the present invention connected to two longitudinal members 12 as would be used to stabilize a spine during spinal surgery. The cross connector 10 comprises a first transverse member 14 having a clamp 16 for affixation to one of the longitudinal members 12, and a second transverse member 18 having a clamp 20 for affixation to the other longitudinal member 12. The first transverse member 14 and second transverse member 18 are locked together by a third clamp 22.

The first transverse member 14 arcs slightly from a proximal end 24 thereof to a distal end 26 thereof and terminates in a ball 28 at the distal end 26. The first clamp 16 comprises an inner curved surface 30 for receiving the ball 28 and a second curved surface 32 for receiving the longitudinal member 12. A first threaded connector 34 passes through an aperture 36 in the first clamp 16 between the first curved surface 30 and second curved surface 32 and engages a threaded aperture 38 in a flanged nut 40. The flanged nut 40 has a curved surface 42 for engaging longitudinal member 12 and an adjacent curved surface 43 for engaging the ball 28. Tightening the threaded connector 34 against the flanged nut 40 clamps the longitudinal member 12 between the flanged nut curved surface 42 and the curved surface 32 on the first clamp 16. It also clamps the ball 28 between the curved surface 42 on the flanged nut 40 and the curved surface 30 on the first clamp 16 thereby inhibiting rotation of the first clamp 16 about the ball 28. Thus the first clamp 16 clamps to the longitudinal member 12 and locks against the ball 28 by tightening a single screw, the threaded connector 34.

The second transverse member 18 arcs slightly from a proximal end 44 thereof to a distal end 46, terminating in the second clamp 20. The second transverse member 18 has a pair of grooves 48 on its upper surface between the proximal end 44 and distal end 46. Conversely, the first transverse member 14 has a pair of splines 50 on a lower surface between its proximal end 24 and distal end 26, which interconnect with the grooves 48 whereby to allow sliding transverse movement between the first transverse member 14 and second transverse member 18.

The third clamp 22 is a C-clamp design which wraps around the first transverse member 14 and second transverse member 18 to compress these two parts into clamping engagement. Accordingly, it comprises a pair of spaced apart arms 52 and 54 connected by an arcing portion 56 to form a transverse aperture 58 therethrough for receiving the first and second transverse members 14 and 18. A screw 60 penetrates a non-threaded aperture 62 on the first arm 52 and enters a threaded aperture 64 on the second arm 54 to tighten the third clamp 22 about the first and second transverse members 14 and 18.

The splines 50 and grooves 48 are formed with a taper lock so that as the clamp 22 tightens the splines 50 engage the grooves 48 with an interference fit. This greatly enhances the holding power of the clamp 22. The arc shape of the transverse members 14 and 18 also enhances the ability of the clamp 22 to resist slippage of the transverse members 14 and 18 as the forces tending to cause slippage will be translated into torque about the arc.

In use, the longitudinal members 12 are fixed to the spine in a traditional manner by means of screws as is known by those of skill in the art. The cross connector 10 connects the two longitudinal members 12 to form a more rigid support for the spine. The sliding movement of the first transverse member 14 with respect to the second transverse member 18 allows the space between the first clamp 16 and second clamp 20 to be adjusted to account for the inevitable variations in spacing of the longitudinal members 12 due to the anatomy of a particular patient. The ball 28 on the first clamp 16 allows polyaxial movement of the first clamp 16 with respect to the rest of the cross connector and can thereby account for longitudinal members 12 which may not be in perfect parallel alignment. It can also allow the surgeon to provide the lowest possible profile for the cross connector 10 by adjusting the distance of the cross connector 10 with respect to the spine by rotation of the first clamp 16 about the ball 28.

Of course, a similar ball joint arrangements could be provided in some other location between the first and second clamps 16 and 20, however, the particular arrangement of the ball 28 as shown inside the first clamped 16 allow a very few number of parts to achieve the clamping action and polyaxial movement.

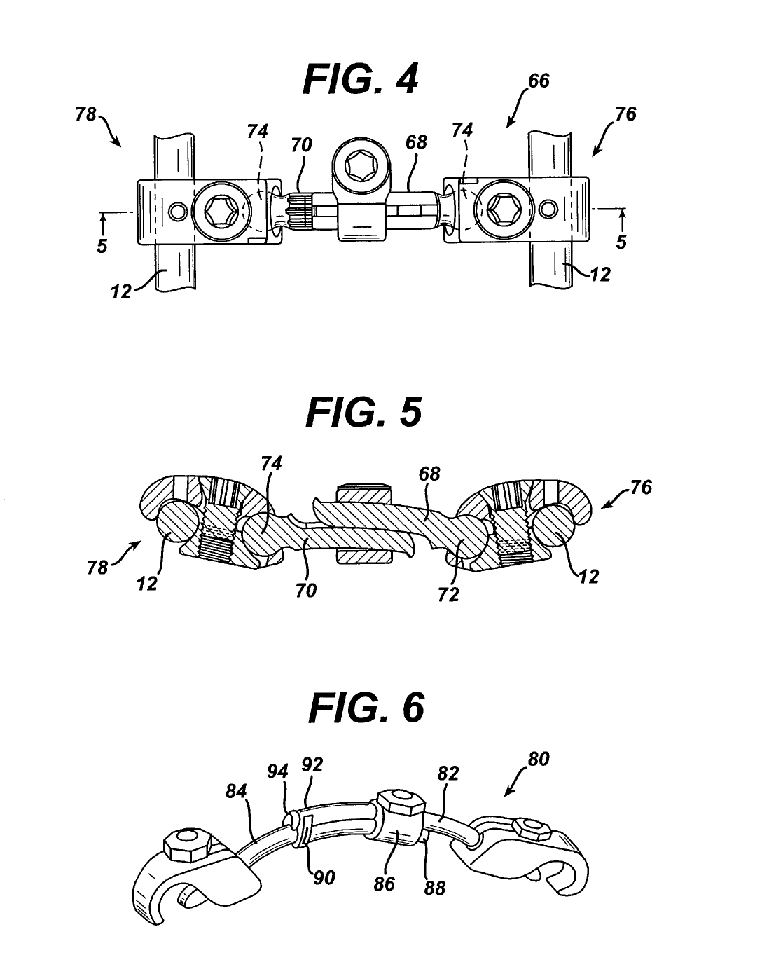

FIGS. 4 and 5 illustrate a cross connector 66 very similar to the cross connector 10 of the previous embodiment. However, the cross connector 66 has a ball joint construction on each clamp. Cross connector 66 comprises first and second transverse members 68 and 70 each terminating in balls 72 and 74, respectively. Affixed to each ball 72 and 74 is a first clamp 76 and second clamp 78, respectively, each of the same construction as the first clamp 16 of the prior embodiment. Accordingly, the cross connector 66 allows polyaxial rotation of the first and second clamps 76 and 78 and enhances the options of the surgeon in disposing this device within a patient's body.

FIG. 6 illustrates a third embodiment of a cross connector 80 according to the present invention. The cross connector 80 is of similar construction to the cross connector 66. However, it employs first and second transverse members 82 and 84 which are longer than in the previous embodiments. To provide an enhanced stability of the first transverse member 82 with respect to the second transverse member 84 the cross connector 80 employs a C-clamp 86 of the same design as the third clamp 22 near a proximal end 88 of the second transverse member 84 and a simple wrap around member 90 at a proximal end 92 of the first transverse member 84. The wrap around member 90 connects to the proximal end 92 of the second transverse member 84 and wraps around the first transverse member 82 thus preventing separation of the two transverse members at this point.

One additional difference between the cross connector 80 and cross connector 66 is the interface between the first and second transverse members 82 and 84. Rather than employ splines, the first transverse member 82 has a circular cross section and the second transverse member 84 has a circular cross section with an under cut lower surface 94 shaped to receive the circular cross section of the first transverse member 82. Of course, a splined interface as in the previous two embodiments could also be substituted therefor.

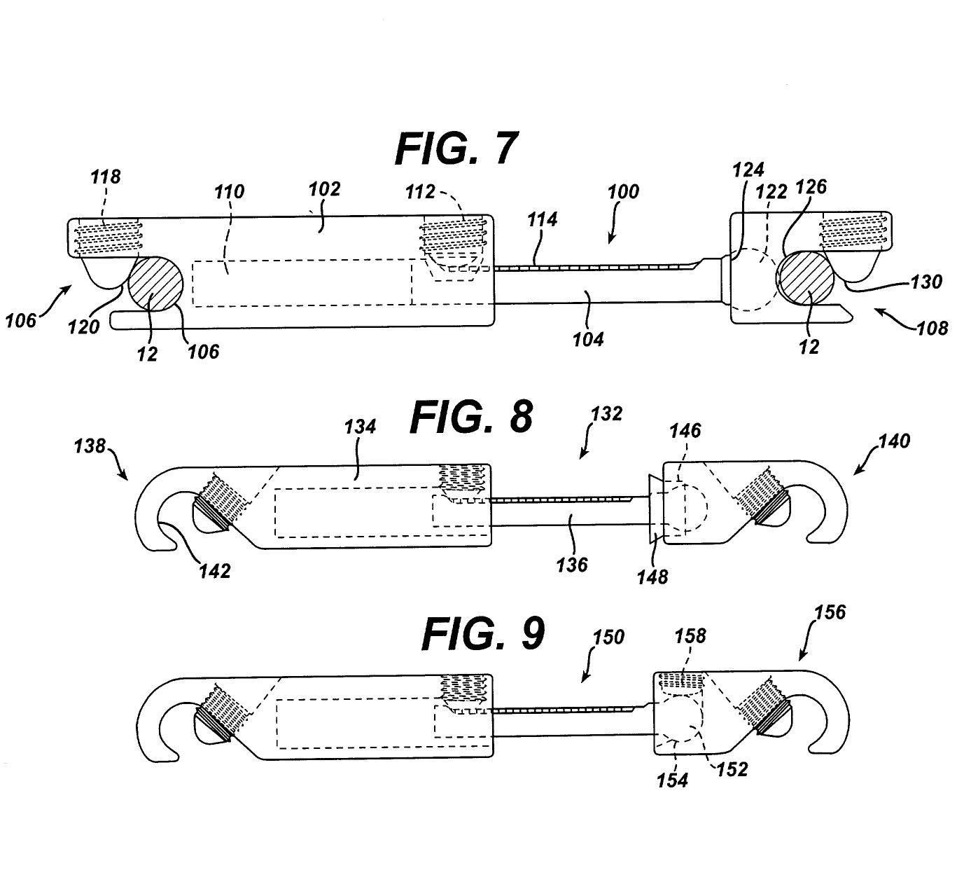

FIG. 7 illustrates a further embodiment of a cross connector 100. The cross connector 100 employs first and second transverse members 102 and 104, having first and second clamps 106 and 108 thereon. The first transverse member 102 has a bore 110 for receiving a portion of the second transverse member 104 in sliding engagement. A set screw 112 penetrates the first transverse member 102 to engage the second transverse member 104 and fix the relative position of the first and second transverse members 102 and 104 with respect to each other. Optional countersinks 114 on the second transverse member 104 where the set screw 112 engages it enhance the purchase between the set screw 112 and the second transverse member 104.

The first clamp 106 comprises an arcuate surface 116 which receives the longitudinal member 12 and a first clamping screw 118 with a camming surface 120 which forces the longitudinal member 12 against the arcuate surface 116 to lock the longitudinal member 12 into the first clamp 106.

A ball 122 sits within a curved enclosure 124 on the second clamp 108 to allow polyaxial rotation of the second clamp 108 about the second transverse member 104. The second clamp 108 also has an arcuate surface 126 and a second clamping screw 128 with a camming surface 130 whereby to force the longitudinal member 12 toward the arcuate surface 126. However, the ball protrudes partially past the arcuate surface 126 such that the force applied by the second clamping screw 128 forces the ball 122 against the curved enclosure 124. Thus, engagement of the second clamping screw 128 both locks the longitudinal member 12 into the second clamp 108 and locks the second clamp 108 to the ball 122.

FIG. 8 illustrates a further embodiment of a cross connector 132 according to the present invention similar to the previous embodiment, comprising first and second transverse members 134 and 136 bearing first and second clamps 138 and 140. Curved surfaces 142 on the first and second clamps 138 and 140 are disposed so that the longitudinal members 12 enter therein at an oblique angle to the first and second transverse members 134 and 136. A ball 144 on the second transverse member 136 sits within a curved enclosure 146 in the second clamp 140 to allow polyaxial movement of the second clamp 140. Rather than engage the longitudinal member 12 as in the previous embodiment, a separate locking nut 148 locks the second clamp 140 to the ball 144. The locking nut threads onto the second clamp 140 and coaxially receives the second transverse member 136. It threads into the curved enclosure 146 to bear against the ball 144 and lock it to the second clamp 140.

FIG. 9 illustrates a further embodiment of a cross connector 150 according to the present invention. It is similar in nearly all aspects to the cross connector 132 of the previous embodiment. A ball 152 is received within an enclosure 154 on a clamp 156. A locking screw 158 enters the enclosure and drives against the ball 152 to lock the clamp 156 thereto.

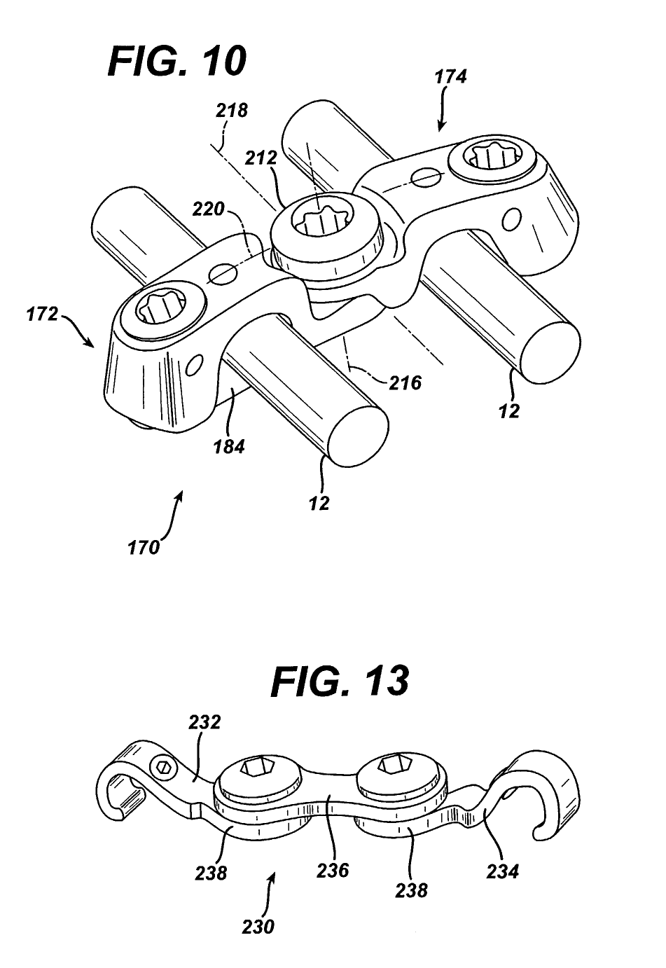

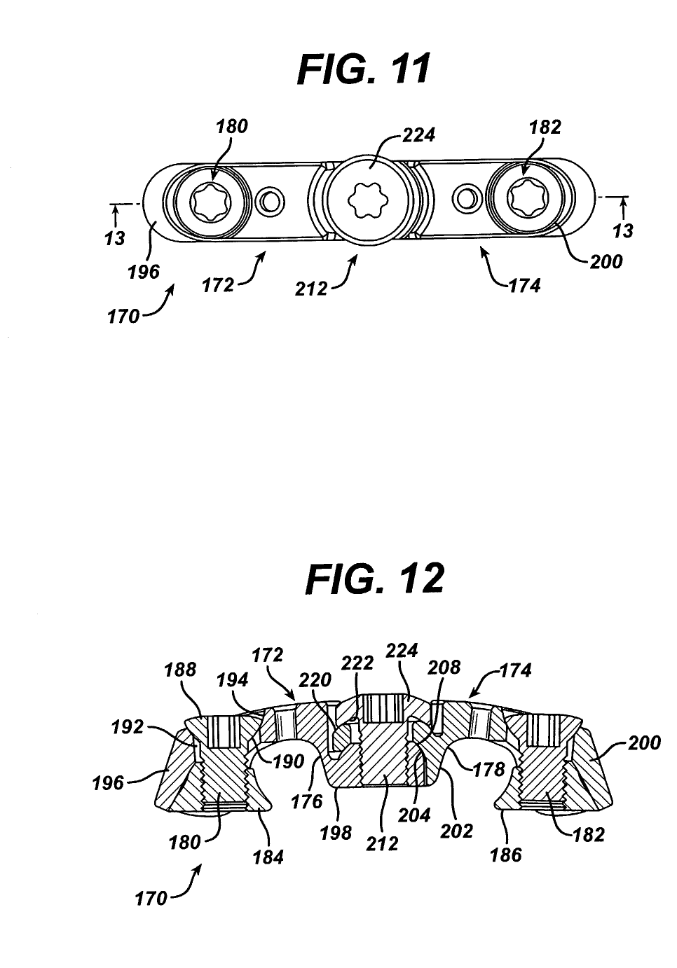

FIGS. 10 to 12 illustrate a further embodiment of a cross connector 170 according to the present invention which is particularly well suited to a rather narrow dimension between the longitudinal members 12. It comprises a first transverse member 172 and second transverse member 174, each of which has respective curved surfaces 176 and 178 for receiving a longitudinal member, and each of which respectively a threaded connector 180 and 182 having a flanged nut 184 and 186. Each threaded connector 180 and 182 has a head 188 having a convex lower surface 190 and passes through an aperture 192 in the transverse members 172 and 174 having a mating concave countersunk surface 194 which allows slight toggling movement of the threaded connectors 180 and 182 prior to tightening. This aids in accommodating various sizes of longitudinal members 12 and is preferably present in each of the embodiments of the invention.

The first transverse member 172 has a distal end 196 and a proximal end 198 and the second transverse member has a distal end 200 and a proximal end 202. The threaded connectors 180 and 182 are disposed at the distal ends 196 and 200. The first transverse member proximal end 198 comprises a convex, preferably spherical, upper surface 204 through which passes a vertical threaded aperture 206. The second transverse member proximal end 202 comprises a mating concave lower surface 208 through which penetrates a non-threaded aperture 210. A threaded connector 212 passes through the aperture 210 to engage the threaded aperture 206.

The mating surfaces 204 and 208 form a ball joint 214 to allow polyaxial motion between the first and second transverse members 172 and 174. A wide degree of freedom is allowed about an axis 216 longitudinally through the threaded connector 212. The aperture 210 is sufficiently wider than the width of the threaded connector 212 to allow a limited degree of freedom about an axis 218 parallel to the longitudinal axis of the longitudinal members 12 and about an axis 220 longitudinal through the cross connector. An upper surface 220 on the second transverse member proximal end 202 is preferably convex and a lower surface 222 on a head 224 of the threaded connector 212 is preferably concave and these surfaces function similarly to the surfaces 204 and 208.

The location of convex and concave surfaces in the ball joint 214 can be reversed and it would be apparent that modifications to the surfaces which nonetheless allow polyaxial movement therebewteen can be substituted therefor.

FIG. 13 illustrates a further embodiment of a cross connector 230 similar in design to the cross connector 170 of FIGS. 10 to 12 in which first and second transverse members 232 and 234 similar to the transverse members 172 and 174 are connected by an intermediate member 236 via ball joints 238 similar to the ball joint 214.

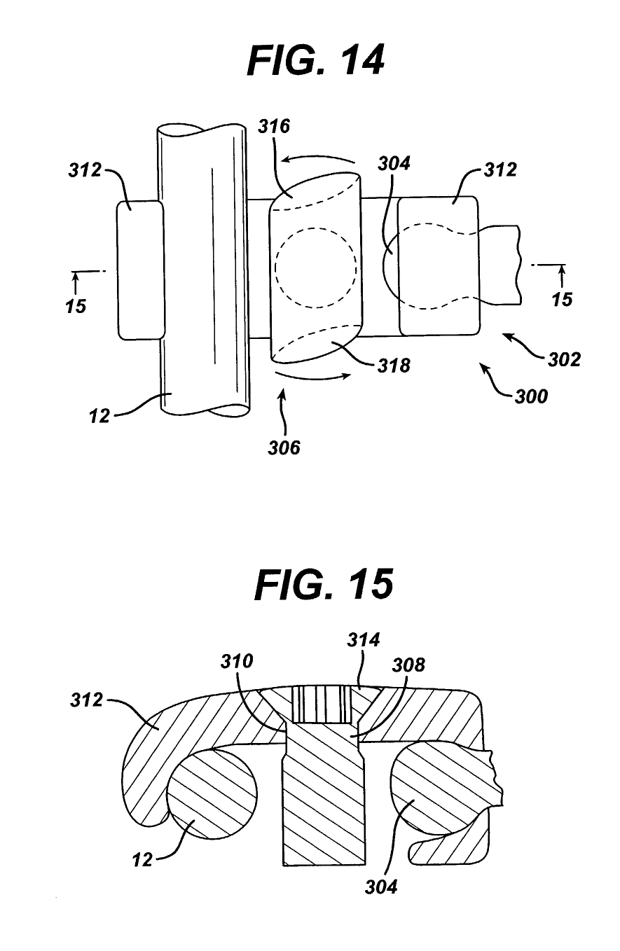

FIGS. 14 and 15 illustrate a further embodiment of a cross connector 300 similar in design to the cross connector 66 of FIGS. 4 and 5. However, it employs a clamp 302 which differs somewhat from the clamp 76 of the cross connector 66. The clamp 302 traps the rod 12 between the camp 302 and a ball 304 (which is similar to the ball 72 of the cross connector 66) with a cam member 306 rather than a screw. The cam member 306 has a stem 308 which protrudes up through an aperture 310 in a body 312 of the clamp 302 and terminates in a screw head 314 or other turning tool engaging surface. As the cam member 306 is rotated via the screw head 314, an outer camming surface 316 cams over the rod 12 forcing it upwardly against the clamp body 312. An inner camming surface 318 acts similarly against the ball 304. One or more detents, not shown, can be provided to more positively lock the cam member 306 into a position in engagement with the rod 12, or out of engagement with the rod 12 (the position as shown in FIGS. 14 and 15). Clamp 302 provides an added advantage of slightly greater clearance for inserting the rod 12 into the clamp 302.

While the invention has been particularly described in connection with specific embodiments thereof, it is to be understood that this is by way of illustration and not of limitation, and that the scope of the appended claims should be construed as broadly as the prior art will permit. Variations and modifications of the disclosed embodiments will occur to those skilled in the art and all such variations and modifications are considered to lie within the scope of the invention as described herein and defined by the claims appended hereto and equivalents thereof. For instance, curved surfaces are disclosed for contact with the balls and longitudinal members; however, flat or angled surfaces, especially as are known in the relevant arts, may be substituted therefor.

Further, each of the clamps on the illustrated embodiments employ a positive locking force to grab the longitudinal members 12, nevertheless it is known to merely capture one of the longitudinal members 12 in a hook shaped recess without otherwise clamping the longitudinal member therein, as for instance the clamp 106 without the screw 118. Each of the embodiments employs some means for changing the spacing between the clamps; however, this feature is optional in the present invention.

What is claimed is:

1. A cross connector for linking longitudinal members engaged to a spine, the cross connector comprising:
a first connector for attaching to a first one of the longitudinal members;
a second connector for attaching to a second one of the longitudinal members;
a linkage between the first and second connectors; and
at least one polyaxial joint located between the first and second connectors.

2. A cross connector according to claim 1, wherein one of the first and second connectors comprises the polyaxial joint.

3. A cross connector according to claim 1 wherein one of the first and second connectors comprises a first clamping member and a second clamping member, and a fastener adapted to hold the first clamping member and second clamping member tightly together whereby to grasp one of the longitudinal members.

4. A cross connector according to claim 3 wherein at least a portion of the at least one polyaxial joint is disposed between the first clamping member and the second clamping member whereby when the fastener holds the first clamping member and second clamping member tightly together, said portion is squeezed to prevent rotation of the at least one polyaxial joint.

5. A cross connector according to claim 4 wherein the first clamping member has a first surface adapted to engage one of the longitudinal members and wherein the second clamping member has a second surface adapted to engage one of the longitudinal members and wherein the fastener is located between the at least a portion of the polyaxial joint and the first and second surfaces.

6. A cross connector according to claim 1 wherein the at least one polyaxial joint comprises a convex surface portion on the linkage and a bearing surface on one of the first and second connectors.

7. A cross connector according to claim 6 wherein the convex portion is spherical.

8. A cross connector according to claim 6 wherein one of the first and second connectors comprises a first clamping member and a second clamping member, and a fastener adapted to hold the first clamping member and second clamping member tightly together whereby to grasp one of the longitudinal members and wherein the convex portion is disposed between the first clamping member and the second clamping member.

9. A cross connector according to claim 1 wherein the linkage is curved whereby to arch over a patient's spine.

10. A cross connector according to claim 9 wherein the linkage comprises a first transverse member and a second transverse member connected in sliding relationship to each other whereby to alter a length of the linkage.

11. A cross connector according to claim 10 wherein one of the first and second transverse members bears at least one groove and the other of the first and second transverse members bears at least one spline slidably received within said groove and wherein the spline fits within the groove with an interference fit when compressed together whereby to prevent sliding of the first and second transverse members with respect to each other.

12. A cross connector according to claim 1 wherein the polyaxial joint comprises a curved surface connected to one of the first connector and second connector and a mating surface thereto connected to the other of the first connector and second connector, the curved surface and mating surface being adapted for movement in three degrees of freedom over one another.

13. A cross connector according to claim 12 wherein the curved surface comprises a convex surface and the mating surface is a complementary concave surface.

14. A cross connector according to claim 13 and further comprising a first threaded aperture penetrating the convex surface, a second aperture through a portion of the cross connector bearing the concave surface and a threaded connector passing through the second aperture and threading into the first aperture to fix the convex surface to the concave surface.

15. A cross connector according to claim 14 wherein the second aperture is wider than the threaded connector.

16. A cross connector for linking longitudinal members engaged to a spine, the cross connector comprising:
a first connector for attaching to a first one of the longitudinal members;
a second connector for attaching to a second one of the longitudinal members; and
a linkage between the first and second connectors;
wherein the linkage is curved whereby to arch over a patient's spine and comprises a first transverse member and a second transverse member connected in sliding relationship to each other whereby to alter a length of the linkage.

17. A cross connector according to claim 16 wherein one of the first and second transverse members bears at least one groove and the other of the first and second transverse members bears at least one spline slidably received within said groove and wherein the spline fits within the groove with an interference fit when compressed together whereby to prevent sliding of the first and second transverse members with respect to each other.
at least one polyaxial joint located between the first and second connectors.

18. A cross connector according to claim 1 wherein one of the first and second connectors comprises a camming member which cams over one of the longitudinal members trapping the longitudinal member between the camming member and a surface in the connector.
